(19) United States
(12) Patent Application Publication
(10) Pub. No.: US 2024/0180147 A1
(43) Pub. Date: Jun. 6, 2024

(54) SYSTEM AND SOLUTION FOR IMPROVED WHOLE BLOOD STORAGE

(71) Applicant: Hemerus Medical, LLC, St. Paul, MN (US)

(72) Inventors: John HESS, Seattle, WA (US); Majid ZIA, White Bear Lake, MN (US)

(73) Assignee: Hemerus Medical, LLC, St. Paul, MN (US)

(21) Appl. No.: 18/553,595

(22) PCT Filed: Apr. 7, 2022
(86) PCT No.: PCT/US2022/023844

§ 371 Date: Oct. 2, 2023

Related U.S. Application Data
(60) Provisional application No. 63/171,880, filed on Apr. 7, 2021.

(51) Int. Cl. A01N 1/02; A61M 1/02

(52) U.S. Cl. CPC A01N 1/021; A01N 1/0263; A61M 1/0218; A61M 1/0272

(57) ABSTRACT

A whole blood storage system that includes whole blood leukoreduction with pH optimization for improved RBC storage. Such a system can provide leukoreduced whole blood for field medical use, but would preserve the RBCs and most plasma coagulation activity for at least about 2 weeks, or about 3 weeks, or about 4 weeks, or about 5 weeks or about 6 weeks so that the units would not go to waste.

### RELATED APPLICATIONS

This application is an International PCT Application claiming priority to U.S. Provisional Patent Application No. 63/171,880, filed Apr. 7, 2021, the entirety of which is incorporated by reference herein.

### FIELD OF THE INVENTION

The present invention relates to the storage of whole blood and blood products. The invention also relates to a system for the collection, processing and storage of blood and blood products.

### BACKGROUND OF THE INVENTION

It is known that hemorrhage is the leading cause of preventable death in both military and civilian traumatic injury. There is continued interest in optimizing transfusion practice during massive hemorrhage resuscitation. Given successful use of whole blood (WB) in the military, civilian hospitals have adopted massive transfusion protocols that simulate WB transfusion, by administering plasma, platelets (PLTs), and red blood cells (RBCs) in equal ratios. For patients with life threatening hemorrhage, resuscitation with blood products is essential in addition to hemorrhage control.

Many methods have been described for blood collection, processing and storage for transfusion. CPDA-2 is a storage solution developed by the US Army (Sohmer P R, Moore G L, Beutler E, Peck C C. In vivo viability of red blood cells stored in CPDA-2. Transfusion. 1982 November-December; 22(6):479-484.). It was developed and tested in humans and worked well, but was never licensed or sold. A variety of whole blood leukoreduction filters exist, and many are currently FDA licensed in the US.

First generation whole blood and red blood cell leukoreduction filters were primarily developed for reducing leukocytes in whole blood or red blood cell products. These filters not only reduced leukocytes but also reduced platelet content of blood and blood products.

The next generation of leukoreduction filters enable the leukoreduction of whole blood while substantially sparing platelets and providing a means for preparation of important leukoreduced therapeutic products (Red Blood Cells (RBC), Platelet Rich Plasma (PRP), Platelet Poor Plasma (PPP), and Platelets). An example of such a system is IMUFLEX® WB-SP by Terumo Corporation. For reference, an overview of the instructions is hereby provided in FIG. 2 for use of the platelet sparing leukoreduction system.

This system uses a typical anticoagulant, CPD, for the collection of whole blood and uses a platelet sparing filter to produce a leukocyte reduced whole blood component. The filtration system includes a bypass to substantially drain the entire collected whole blood component through the filter and to prepare a substantially air-free leukoreduced CPD whole blood component. The platelet rich whole blood component may further be further separated into blood components including red blood cells (RBCs) in additive solution.

Blood storage systems are typically acidic at pH 5.5 to prevent the dextrose they contain from caramelizing when they are autoclaved to sterilize them. Adding extra alkaline constituents to raise the pH improves metabolism. Bicarbonate is particularly useful in this regard because it is nontoxic, breaking down into water and CO₂ and a buffer.

Adding sodium bicarbonate to raise the pH closer to but less than 7.2 and buffer the acid produced by glycolysis was developed by Hess & Greenwalt and is the subject of the following patents, all of which are hereby incorporated by reference to the extent permissible by law:
U.S. Pat. No. 6,150,085, Hess & Greenwalt, Prolonged red blood cell storage and composition (9- & 10-week RBC storage solutions).
U.S. Pat. No. 6,447,987, Hess & Greenwalt, Prolonged red blood cell storage (11-week).
U.S. Pat. No. 8,709,707 Hess & Greenwalt, “Compositions substantially free of sodium chloride and methods for the storage of red blood cells”
U.S. Pat. No. 9,314,014, Hess & Greenwalt. Compositions and methods for the storage of red blood cells.

Nonetheless, there remains a need for methods and systems to store whole blood and components thereof, including red blood cells.

### SUMMARY OF THE INVENTION

Described herein, is a whole blood storage system that includes whole blood leukoreduction with pH optimization for improved RBC storage. The system, in some embodiments, includes a two-component anticoagulant system that not only enables sterilization of the contents without degradation but also simplifies the design, components, operations, and overall cost of the collection and processing system.

Such a system can provide leukoreduced whole blood for field medical use by preserving the RBCs, plasma, platelets and in effect maintaining effective oxygen delivery and coagulation activity of the whole blood for weeks, in some embodiments, for about 2 weeks, or about 3 weeks, or about 4 weeks, or about 5 weeks, and subsequent possibility of preparation of components such that highly valuable blood units would not go to waste.

Accordingly, in a first aspect, the present invention is directed toward a whole-blood storage utilizing two additives, at least one of which is inherently an anti-coagulative agent. One additive may be a standard anti-coagulant including, without limitation, CPD, CP2D, CPDA-1, and CPDA-2, and the second additive may include components that raise the pH of the first additive. For example, the second additive may include, among other components, a bicarbonate ion-providing component such as sodium bicarbonate. In another non-limiting example, the first additive may include citric acid, sodium citrate, and/or dextrose and the second additive may include phosphate, bicarbonate and adenine. Other constituents and various separation of the constituents into the first additive and the second additive are possible to provide improved storage and therapeutic benefits. The system may, in some embodiments, include first and second additive bags for holding the first and second additives. In some embodiments, the first additive bag is suitable for the storage of whole blood and/or blood plasma and/or red blood cells (RBC).

### BRIEF DESCRIPTION OF THE DRAWINGS

The above, as well as other advantages of the present invention, will become readily apparent to those skilled in the art from the following detailed description when considered in the light of the accompanying drawing in which:

FIG. 1 is a schematic diagram showing a non-limiting system for collection and storage of blood and blood products in accordance with a non-limiting embodiment of the invention; and

FIG. 2 is known and shows an overview of instructions by Terumo Corporation for use of the known platelet sparing leukoreduction system.

### DETAILED DESCRIPTION OF THE PREFERRED EMBODIMENTS

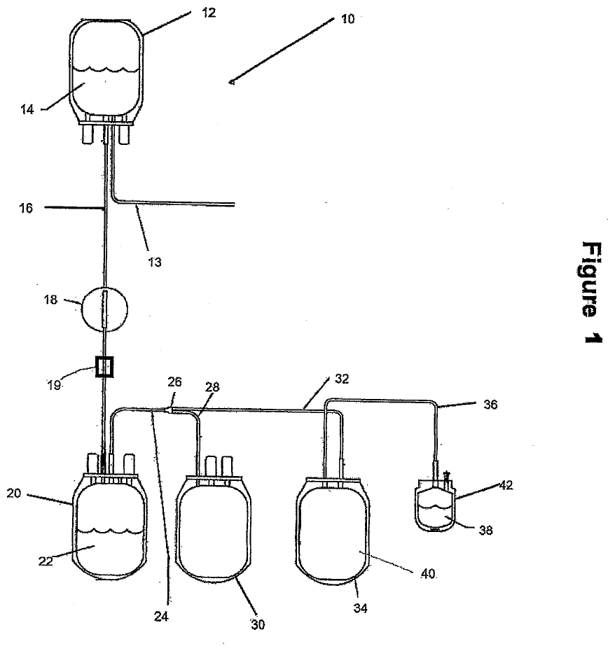

FIG. 1 shows a schematic showing the system for collection and storage of blood and blood products in accordance with an embodiment of the invention.

In a preferred embodiment of the present invention, a whole blood storage system 10 includes a blood donor bag 12. A blood input system 13 can allow for blood to be brought in from a donor. A first additive 14 is contained within donor bag 12. The additive 14 may have anti-coagulating and/or storage properties.

The donor bag 12 is connected by a line 16 to an additive bag 20, with a second additive 22 contained within the bag 20. Optionally, breakaway cannula 19 are either incorporated within the bags or may be placed in line within lines such as 16 to initiate fluid flow between components at desired processing times. In some embodiments, a leukoreduction filter 18 is present in the line between the donor bag 12 and the additive bag 20. At least one, and in a preferred embodiment, both, of the donor bag 12 and the additive bag 20 are suitable for the storage of whole blood and/or blood plasma and/or red blood cells (RBC).

The second additive 22 may or may not have anti-coagulating properties on its own, but in a preferred embodiment of the present invention the combination of the first and second additives has anti-coagulative properties and superior storage capability for blood storage than those of the first additive alone.

The second additive 22 may be added to the first additive 14 prior to or after whole blood is collected in donor bag 12. The purpose of the additives is to improve storage capability of the collected and to be processed whole blood. The volume and anticoagulant content are selected to provide optimum nutrients for storage of blood, a means of prevention of degradation products from sterilization of blood bag set by steam sterilization, and to reduce cost of the set by eliminating bypass and/or soft filter requirements to maximize post filter blood recovery and minimizing excess air present in blood or blood components for storage.

In further embodiments of the present invention, whole blood that that is collected by the system of the present invention can be stored for up to about 5 weeks, and can subsequently be further processed into red blood cells (RBCs) and plasma, and at least the RBCs can be stored for further periods and at least 6 weeks from whole blood collection.

Towards that end, an exit line 24, as shown coming from additional bag 20 can facilitate transfer and preparation blood components after separation of whole blood into blood components (typically a centrifuge, although other means for separating RBCs from plasma could be used in accordance with embodiments of the present invention). This device 26 can then separate plasma (for example, platelet-rich plasma) which proceeds through a line 28 into a plasma bag 30 and RBCs which proceed through a RBC line 32 into a RBC bag 34. While a centrifuge is preferred and discussed herein, other means for separating the components of whole blood can be used in accordance with the present invention without affecting the scope of the present invention. The platelet rich plasma may be separated into Platelet Poor Plasma (PPP) and Platelet products,

In accordance with additional embodiments of the present invention, a further additive 40 could be stored in bag 34 for where to be combined with separate RBCs from Whole Blood. Although RBCs may be transferred to bag 34, it would be preferred to add contents of bag 34 into bag 20 after whole blood separation and transfer of plasma to plasma bag 30.

Additionally, a further additive 42 could be stored in an additional additive bag 38 for transfer through line 36 into bag 34 for use alone or with optional additive 40 such as Red Blood cell additive AS-7 which is a two-component additive for red blood cells.

Standard technology for the collection of blood can be utilized with embodiments of the present invention. For example, a needle with a needle protector can tie into line 13 into the donor bag 12, with a bypass line connecting to a sampling bag and a sampling port.

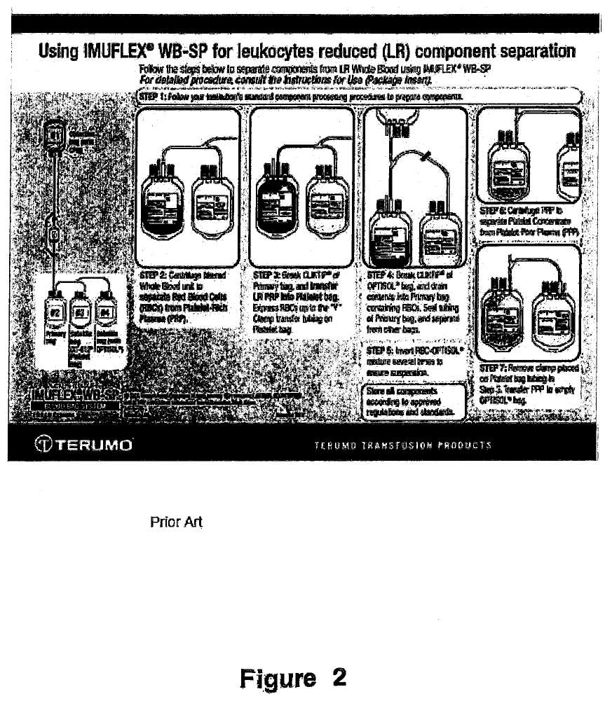

FIG. 2 shows an overview of instructions by Terumo Corporation for use of a known platelet sparing leukoreduction system.

A preferred embodiment of the present invention utilizes a conventional primary blood collection bag containing CPDA-2 anticoagulant in a volume about 1/7 that of the anticipated blood draw (63 mL for a conventional “pint” draw of 450 mL or 70 mL for a modern 500 mL draw). The primary bag can be connected to a secondary bag appropriate for blood storage and administration by tubing with an integral whole blood leukocyte reduction filter and long enough to be heat sealed into about 8-12 segments about 2-4 inches long for blood typing. The secondary storage bag can contain about 40 mL of sterile sodium bicarbonate solution 12 mEq in sterile water for injection.

CPDA-1 is approved for storage of blood for 35 days (5 weeks). CPDA-2 has not entered into use in the US, but references show acceptable data based on the standard required in the 1980s, which requires mean in vivo recoveries of at least 70%. At that time of the development of CPDA-2 leukoreduction was not common and leukoreduction might have helped to obtain this standard, but it would have removed valuable platelets.

Standard Leukoreduction filters capture platelets but there are currently platelet sparing filers available such as those used in IMUFLEX WB-SP blood storage system by Terumo (code: 1BB*LGQ506A6) which may be suitable for use in embodiments of the present system.

It is also possible to make a similar system in which citric acid, sodium citrate, and dextrose are the primary anticoagulant in the donor (first) bag and phosphate, bicarbonate and adenine are in the second bag with relatively less volume in the first bag and more of the volume in the second, but this changes the “form and feel” of the collection set compared to standard blood bags, which is not necessarily advantageous for a product designed primarily for emergency use.

As in standard blood collection, venous blood, typically from the arm of the donor, drains into the anticoagulant in the donor bag and is mixed during collection by gentle agitation. If platelet product is of interest, the whole blood is held and processed at room temperature. Otherwise, whole blood may be stored in refrigerated storage (typically 1-6° C.) until used or processed into components. The preference will likely be to hold the blood at room temperature prior to processing into components as better platelet yields may be possible.

Processing may provide running, for example, about 40 mL of the second additive (e.g. bicarbonate solution) in the secondary bag through the filter to thoroughly wet the filter by hanging the system with the secondary bag on top. When substantially all of the solution is in or through the filter the system is inverted and the whole blood is drained from the primary bag through the filter into the secondary bag and the secondary bag is mixed, the line filled with whole blood, segmented by heat sealing, and the bag is then placed in refrigeration.

The volume of fluid in the second bag may be varied from about 15 to 60 mL to insure adequate wetting of the filter. Referring to FIG. 1, the expected volume of second additive 22 in the additive bag 20 should be at least the holdup volume of the filter. The concentration of second additive 22 (e.g. bicarbonate) in the secondary bag 20 may be varied from about 5 to 60 mEq to insure that the starting pH of blood storage is approximately 7.2 so that ATP metabolism in not disturbed. In some embodiments, a concentration of about 12mEq is used. (See Hess J R, Hill H R, Oliver C K, Lippert L E, Greenwalt T J. Alkaline CPD and the preservation of RBC 2,3-DPG. Transfusion. 2002 June; 42(6):747-752.)

Therefore, in a preferred embodiment of the present invention a whole-blood storage system that includes whole blood leukoreduction filter with pH optimization for improved red blood cell (RBC) storage, comprises a first additive and a second additive, wherein upon the first additive and the second additive being combined with whole blood the coagulation capability of the whole blood is maintained for at least 3 weeks.

In some embodiments, the first additive comprises an anti-coagulating agent. In some embodiments, the second additive comprises an anti-coagulating agent. In some embodiments, each of the first additive and the second additive comprises an anti-coagulating agent.

In some embodiments, upon the first additive and the second additive being combined with whole blood, the whole blood can be preserved for at least 2 weeks, or at least 3 weeks, or at least 4 weeks, or at least 5 weeks, and red blood cells can be preserved for at least 2 weeks, or at least 3 weeks, or at least 4 weeks, at least 5 weeks, or at least 6 weeks.

As used herein, by “preserve” is meant that the indicated cells meet the criteria for being preserved after being stored for the indicated time. The time will differ for the type of cell being stored. When the cells being stored are red blood cells (RBCs), the RBCs are said to be preserved for 6 weeks (i.e, 42 days) when the RBCs have a level of hemolysis below about 1.0% with 95% confidence that at least 95% of the population estimate will be less than 1% after 42 days of storage. When the cells being stored as whole blood (WB), the WB is said to be preserved for 5 weeks (i.e., 35 days) based on red blood cell quality parameters which are well defined in regulation and evidence of procoagulant activity as measured in vitro by measures such as thromboelastography (TEG) with quality parameters such as Maximum Amplitude as no regulatory standards have been promulgated to date.

In one embodiment, the first additive comprises at least one of citric acid, sodium citrate, and dextrose.

In some embodiments, the first additive comprises CPD, CP2D, CPDA-1 or CPDA-2.

In some embodiments, the second additive comprises a component that provides phosphate, a component that provides bicarbonate, and/or adenine. For example, in some embodiments, the second additive comprises sodium bicarbonate. In some embodiments, the second additive comprises sodium phosphate. In some embodiments, the second additive comprises adenine.

In some embodiments, the first and second additives are stored separately prior to use. In some embodiments, the first and second additives are mixed after sterilization of the blood collection set. In some embodiments, the sterilization is by autoclave in which sterilization medium is a mixture of steam and air.

In some embodiments, the first additive is stored in a first or donor bag, and the second additive is stored in a second or additive bag, wherein at least one of the first and second bags is suitable for whole blood storage.

In some embodiments, the system comprises at least one integral leukoreduction filter and/or platelet sparing filter.

In some embodiments, whole blood stored in this system for up to 2 weeks, or up to 3 weeks, or up to 4 weeks, or up to 5 weeks can then be processed into RBCs in separate additive solution(s) for preservation of red blood cells in additive solutions for at least 2 weeks, or at least 3 weeks, or at least 4 weeks, or at least 5 weeks, or at least 6 weeks from whole blood collection (or phlebotomy).

In a method of storing whole blood in accordance with the present invention, the method comprises using a whole blood storage system that includes whole blood leukoreduction with pH optimization for improved red blood cell (RBC) storage, the storage system comprising a first additive bag containing a first additive and a second additive bag containing a second additive. The method includes the steps of adding whole blood to be processed into the first additive bag, and before, at the same time as, or after adding the whole blood to be processed into the first additive bag, transferring the second additive from the second additive bag to the first additive bag. The whole blood partially or completely combined and mixed with the at least one of the two additives (for example, at least with the first additive) may be leukoreduced by passing the whole blood mixture (with the two additives) through the leukoreduction filter (for example, a leukoreduction filter that is platelet sparing) to produce leukoreduced whole blood. Leukoreduced whole blood may be stored for transfusion or processed into blood components by routinely known methods. The additives combine to preserve the RBCs and most coagulation capability for at least 2 weeks or at least 3 weeks and then the whole blood can be stored for a period of at least 3 weeks, or at least 4 weeks, or at least 5 weeks after collection or phlebotomy. In some embodiments, at least one leukoreduction filter is included in the system, which leukoreduction filter may be platelet sparing. The first additive, in some embodiments, comprises an anti-coagulating agent. Subsequent to the storing of the whole blood, RBCs can be separated from the whole blood, resulting in (or deriving) RBCs and plasma, which can then be stored for additional time.

In some embodiments, the present invention is designed to optimize whole blood collection for use in blood centers supporting field medical operations or processed into blood components for component therapy. Additionally, it could be used in the field in support of walking blood banks in remote locations such as distant military theaters or remote island territories.

In a further embodiment of the present invention, a composition is used to facilitate the shift in transfusion support from blood component therapy to whole blood therapy, so as to, reduce weight and complexity of transfusion.

Component therapy is the separation of donated WB into its component parts of red blood cells, plasma, and platelets. For example, this allows the RBCs, plasma, and platelets from a single donation to support the red cell needs of a patient with anemia, the plasma needs of a patient undergoing plasma exchange for myasthenia gravis, and the platelets support a child with leukemia undergoing chemotherapy. It is in this sense that blood collectors say, “One donation can save three lives.” However, acutely injured individuals are bleeding most commonly and need all three of the components to address their needs for blood volume replacement, oxygen-carrying RBCs, and procoagulant plasma and platelets. Getting three separate components together is difficult now as the storage requirements for the individual blood components have diverged. Today, RBCs are stored in the refrigerator in an extra 100 or 110 mL of additive solution with 450 or 500 ml Whole Blood collection, respectively. Platelets are stored at room temperature with agitation to facilitate their respiration, and plasma is stored frozen and thawed only as needed. The divergent storage requirements means that large-volume blood support is logistically complicated, requiring many bags of many components, each with their own logistical requirements such as freezers, refrigerators, air conditioning, and agitators. Building simpler blood support tools based on keeping WB whole, using simplified blood collection sets, optimized storage solutions and simple ice-chest storage conditions, can eliminate the need for freezers and air conditioning, reduce the weight of collection sets and processed blood products, and reduce blood transport box weight and energy requirements.

Thus, in some embodiments, the invention provides an anticoagulant formation that accomplishes the objectives of a) prevention of activation of the clotting cascade; and b) preservation of quantitative and qualitative levels of WB components including, for example, during long term storage, such as long-term storage that in which the WB components are stored refrigerated (e.g., between 1-6 degrees Celsius) that may be agitated or may not be agitated during storage.

The system can include a 16G needle connection to a primary collection, conventional, plastic bags (blood containers) made of polyvinyl chloride (PVC) plastic with di(2-ethylhexyl) phthalate (DEHP) or PVC with non-ortho-phthalate plasticized containers in a closed system. This primary bag is connected to a platelet sparing leukoreduction filter (Terumo, Lakewood, CO) with about 40 mL dwell and a secondary blood container bag (PVC/DEHP) with two output ports and about 50 mL of the novel storage solution. Primary Bag contains 70 mL of citrate/phosphate/dextrose (CPDA-1, USP). Blood will be collected at a preferred ratio of 1.4:10 in the standard anticoagulant CPDA-1.

In one embodiment, a whole blood (WB) collection system may utilize two solutions for preservation of WB or components thereof (e.g., RBCs). In one non-limiting example, the first solution (which may be either the first or second additive as used herein) may be a standard CPDA-1 anticoagulant (for example, the CPDA-1 product sold by Fenwal, Inc., Lake Zurich, IL, USA). In one embodiment, a second solution (which may be either the first or second additive as used herein) may be a formulation containing, among other components, one or more of the following: a bicarbonate ion providing component such as sodium bicarbonate, a phosphate ion providing component such as sodium bisphosphate (Na2HPO4), a sugar such as dextrose or glucose or sucrose, a sugar alcohol such as mannitol or sorbitol, a salt such as sodium acetate or magnesium chloride or magnesium citrate, a nucleobase containing component such as adenine or guanosine, and an amino acid or derivative thereof such as carnitine or methionine. It shall be understood that such a second solution (which may be a first additive or a second additive as the terms are used herein) may include two of the same components, for example, two sugars such as dextrose and glucose. In some embodiments, the components may be present within the range amounts shown below in Table 1.

| | Concentration in about |
|---|---|
| Component | 50 mL of second solution (mM) |
| Bicarbonate | 40-120, such as between about 60 to 100 mM |
| Phosphate | 10-30, such as between about 15 to 25 mM |
| Mannitol | 55-165, such as between about 90-130 mM |
| Acetate | 50-150, such as between about 80 to 120 mM |
| Salt | 0 to 10, such as between about 6 to 8 mM |
| Nucleobase | 0 to 4, such as between about 2 to 3 mM |

All these components are known chemicals that are chemically stable and physiologically active. The formulation has been designed to maintain the metabolic needs of red cells and platelets with potential storage shelf-life of up to 42 days.

In accordance with the provisions of the patent statutes, the present invention has been described in what is considered to represent its preferred embodiments. However, it should be noted that the invention can be practiced otherwise than as specifically illustrated and described without departing from its spirit or scope.

What is claimed:

1. A whole-blood storage system that includes whole blood leukoreduction filter with pH optimization for improved red blood cell (RBC) storage, comprising:
a first additive; and
a second additive; wherein
upon the first additive and the second additive being combined with whole blood the coagulation capability of the whole blood is maintained for at least about 3 weeks or at least about 4 weeks or at least about 5 weeks after combination.

2. The whole-blood storage system according to claim 1, wherein at least the first additive comprises an anti-coagulating agent.

3. A whole-blood storage system that includes a whole blood leukoreduction filter with pH optimization for improved red blood cell (RBC) storage, comprising:
a first additive; and
a second additive;
upon the first additive and the second additive being combined with whole blood, the whole blood can be preserved for at least about 2 weeks, or about 3 weeks, or about 4 weeks, or about 5 weeks after combination and red blood cells for at least about 2 weeks, or about 3 weeks, or about 4 weeks, or about 5 weeks or about 6 weeks after combination.

4. The whole-blood storage system according to claim 3, wherein the first additive comprises at least one of citric acid, sodium citrate, and dextrose.

5. The whole-blood storage system according to claim 3, wherein the first additive comprises CPDA-1 or CPDA-2.

6. The whole-blood storage system according to claim 3, wherein the second additive comprises at least one of phosphate, bicarbonate and adenine.

7. The whole-blood storage system according to claim 6, wherein the second additive comprises sodium bicarbonate.

8. The whole-blood storage system according to claim 3, wherein the first and second additives are stored separately prior to use.

9. The whole-blood storage system according to claim 8, wherein the first additive is stored in a first bag, and the second additive is stored in a second bag, wherein at least one of the first and second bags is suitable for whole blood storage.

10. The whole-blood storage system according to claim 9, wherein the first bag is suitable for whole blood storage.

11. The whole-blood storage system according to claim 10, wherein the first bag and the second bag are separated by at least one integral leukoreduction filter.

12. The whole-blood storage system according to claim 3, wherein neither the first additive nor the second additive alone will preserve whole blood for more than about 5 weeks or more than about 6 weeks.

13. The whole-blood storage system according to claim 9, wherein the at least one of the first and second bags that is suitable for whole blood storage is configured to receive the other of said first and second additives and whole blood whereupon said whole blood can be preserved for at least about 3 weeks, or at least about 4 weeks, or at least about 5 weeks or at least about 6 weeks after combination.

14. The whole-blood storage system according to claim 3, wherein whole blood stored in this system for up to 5 weeks after combination can then be processed into RBCs in separate additive solution for preservation of at least about 3 weeks, or at least about 4 weeks, or at least about 5 weeks or at least 6 weeks after combination.

15. The whole-blood storage system according to claim 3, wherein the stored whole blood is platelet sparing.

16. The whole-blood storage system according to claim 9, wherein the at least one of the first and second bags that is suitable for whole blood storage is configured to receive the other of said first and second additives and whole blood whereupon said whole blood can be preserved for at least about 3 weeks, or at least about 4 weeks, or at least about 5 weeks after combination.

17. A method of storing whole-blood with a whole-blood storage system storage system that includes whole blood leukoreduction with pH optimization for improved red blood cell (RBC) storage, the storage system comprising:
a first bag containing a first additive; and
a second bag containing a second additive;
the method comprising:
adding whole blood to be collected into the first bag,
before or after adding the whole blood into the first bag,
transferring the second additive from the second bag to the first bag;
wherein the additives combine to preserve the RBCs and most coagulation capability for at least about 3 weeks or at least about 4 weeks or at least about 5 weeks; and
storing the whole blood for a period of up to about 2 weeks, or about 3 weeks, or about 4 weeks, or about 5 weeks or about 6 weeks.

18. The method according to claim 17, wherein the storage system further comprises at least one leukoreduction filter, which leukoreduction filter may be platelet sparing.

19. The method of claim 17, further comprising subsequent to the storing of the whole blood for a period of up to about 2 weeks, or about 3 weeks, or about 4 weeks, or about 5 weeks, separating RBCs from the whole blood resulting in RBCs and plasma.

20. A method of storing red blood cells separated from whole blood, with a storage system storage system that includes whole blood leukoreduction with pH optimization for improved red blood cell (RBC) storage, the storage system comprising:
a first bag containing a first additive; and
a second bag containing a second additive; the method comprising:
adding whole blood into the first additive bag,
before or after adding the whole blood to be stored into the first bag, transferring the second additive from the second bag to the first bag;
wherein the additives combine to preserve the RBCs and most coagulation capability for at least about 3 weeks or at least about 4 weeks or at least about 5 weeks; the method further comprising separating the RBCs from the whole blood, and storing the RBCs for a period of up to about 2 weeks, or about 3 weeks, or about 4 weeks, or about 5 weeks, or about 6 weeks after separating the RBCs from the whole blood.
